(19) United States
(12) Patent Application Publication
(10) Pub. No.: US 2005/0202404 A1
(43) Pub. Date: Sep. 15, 2005

(54) Method for separating a cell group contained in a sample into individual cells

(76) Inventors: Thomas Wittenberg, Erlangen (DE); Matthias Grobe, Regensburg (DE); Robert Couronne, Erlangen (DE); Heiko Kuziela, Erlangen (DE); Christian Muenzenmayer, Nuernberg (DE); Klaus Spinnler, Erlangen (DE); Paulus Dietrich, Herzogenaurach (DE)

Correspondence Address: GLENN PATENT GROUP, 3475 EDISON WAY, SUITE L, MENLO PARK, CA, 94025, US

(21) Appl. No.: 10/970,300

(22) Filed: Oct. 20, 2004

Related U.S. Application Data
(63) Continuation of application No. PCT/EP02/10200, filed on Sep. 11, 2002.

(30) Foreign Application Priority Data
Apr. 22, 2002 (DE) 10217858.5

(51) Int. Cl. C12Q 1/00; C12Q 1/68; G06F 19/00; G01N 33/48; G01N 33/50

(52) U.S. Cl. 435/4; 702/19; 435/6

(57) ABSTRACT

In a method for separating a cell group contained in an image of a sample into individual cells for a subsequent classification of the sample, wherein the cell group has a plurality of mutually overlapping cells, first a cell nucleus of a first cell which is to be separated from the cell group is selected, wherein the cell nucleus is located adjacent to a cell nucleus of a second cell. The cell plasma of the first cell and the cell plasma of the second cell overlap such that a common cell plasma is formed. Subsequently, a contraction of the common cell plasma between the cell nucleus of the first cell and the cell nucleus of the second cell is determined. At the contraction, a separation of the common cell plasma is performed. Subsequently, an area of the common cell plasma is determined in which the overlapping of the cell plasmas of the two cells is expected. This determined area is then classified to associate individual portions of the same with the cell plasma of the first cell and/or with the cell plasma of the second cell. On the basis of the classified portions, the cell plasma of the first cell obtained by the separation is then completed.

### CROSS-REFERENCE TO RELATED APPLICATION

This application is a continuation of copending International Application No. PCT/EP02/10200, filed on Sep. 11, 2002, which designated the United States and was not published in English.

### BACKGROUND OF THE INVENTION

1. Field of the Invention

The present invention relates to a method for separating a group of cells contained in a sample into individual cells, particularly, the present invention relates to a method for separating a cell group contained in an image of a sample into individual cells for a subsequent classification of the sample, wherein the cell group comprises a plurality of mutually overlapping cells.

2. Description of the Related Art

For the successful treatment of cancerous diseases, early detection and treatment is necessary. This can be achieved by regularly attending cancer screening tests. In such tests, smears of the tissue to be examined are taken, wherein, in the case of an examination for cervical cancer (cervical carcinoma), this is usually done by the “PAP test” named after the Greek physician Dr. George Papanicolaou, who introduced this method in 1942. This gynecological smear of the cervix, i.e. the neck of the uterus, or cell specimens obtained from other examinations, generally referred to as cytological specimen, must be classified. For this purpose, the cytological specimens are placed onto a slide, dyed and assessed under the microscope by a cytologist.

For the objective diagnostic assistance for such an expert, image processing programs have recently been used with which the cells of a specimen are automatically segmented and classified based on the morphometric properties of cell nucleus and cell plasma, such as extension of the nucleus and the plasma, form of the nucleus and the plasma, relative size of nucleus and plasma, etc., as well as based on the texture of the chromatin structure in the cell nucleus. The image processing programs employed here, however, only allow a reliable segmentation of the cells of a specimen, when these cells of the specimen occur individually.

While, due to their training, cytologists are capable of implicitly separating cell plasmas that overlap each other up to a certain extent to subsequently make a diagnosis into healthy, inflammatory, dysplastic or diseased cells, automatic methods for segmenting and separating such overlapping cells are not known.

Commonly, either only individual cells are used for automatized classification approaches or a manual cell separation is added in between the automatized steps.

The disadvantage of the first approach, using only individual cells, is that no further attention is paid to the overlapping cells, i.e. they are discarded, so that the important information for the classification of a sample contained also in the overlapping cells is lost.

Although this loss of information is avoided in the second approach, the disadvantage is that a fully automatic pre-assessment of a sample is not possible. As soon as an overlapping is found or likely, it is necessary to call on a human examiner for the separation. After the separation, the automatic method proceeds.

The presence of individual cells, however, is generally the exception in cytological specimens. Rather, depending on the type of specimen, up to 80% of all cell plasmas of a specimen will overlap each other in the cytological specimens/samples. Thus it is necessary for nearly every classification of cytological specimens to provide a manual cell separation or to abandon the information contained in these overlapping cells.

### SUMMARY OF THE INVENTION

It is the object of the present invention to provide a method that detects an overlapping of cells or cell plasmas and segments and separates the individual overlapping cells in order to fulfil the requirements for an automatic classification/assessment of a sample.

The present invention provides a method for separating a cell group contained in an image of a sample into individual cells for a subsequent classification of the sample, wherein the cell group has a plurality of mutually overlapping cells, having the following steps: (a) selecting a cell nucleus of a first cell which is to be separated from the cell group, wherein the cell nucleus of the first cell is located adjacent to a cell nucleus of the second cell, wherein the cell plasma of the first cell and the cell plasma of the second cell overlap each other such that a common cell plasma is formed; (b) determining a contraction of the common cell plasma between the cell nucleus of the first cell and the cell nucleus of the second cell; (c) separating the common cell plasma at the contraction; (d) determining an area of the common cell plasma in which the overlapping of the cell plasmas of the first cell and the second cell is expected; (e) classifying the determined area to associate individual portions of the same with the cell plasma of the first cell and/or the cell plasma of the second cell; and (f) completing the cell plasma of the first cell obtained in step (c) based on the classified portions.

According to a preferred embodiment of the present invention, the method is based on an image which has been generated from a cytological specimen and/or a sample. The image was generated and digitalized, for example, by means of a microscope, wherein each image has a resolution dependent on the capturing device (camera, objective, etc.), such as 1000×700 pixel. The image was captured either in the transmitted light modality or in the fluorescence modality, wherein other known capturing modalities may also be used. According to another embodiment, a plurality of images is used instead of one image, which are registered with each other, and which were generated in different capturing modalities. The different capturing modalities include, for example, capturing an image in a transmitted light modality and capturing a further image in a fluorescence capturing modality. Alternatively, the images may both be generated in the fluorescence capturing modality, but with different parameters regarding the fluorescence.

On the basis of the images thus generated, an automatic segmentation or separation of cell groups into individual cells is performed using the inventive method, which may then form the basis for an automatic further processing for the classification of the cytological sample.

The advantage of the present invention is thus that adding a manual cell separation as well as the work and loss of time connected therewith can be avoided, while at the same time, the information for the classification of cytological specimens contained in the cell groups, also referred to as cell clusters, is no longer lost, but is used for their classification to allow putting the results of the classification on a broader basis of the cells contained in the cytological specimen. This results in the advantage of an improvement in the reliability of the classification of the specimens subsequently performed.

According to another preferred embodiment, the inventive method also includes the preparatory steps required to detect, from a picture (one image or several images) of the sample, one or more cell groups which are then separated into individual cells according to the invention. According to this embodiment, first a detection and segmentation of cell nuclei is performed based on an image of the sample to generate a list of cell nuclei. Next, a detection and segmentation of cell plasmas is performed based on the image of the sample to generate a list of the cell plasmas. The cell nuclei are associated with the pertinent cell plasmas, and, based on the number of cell nuclei associated with a cell plasma, the method detects whether the combination is a cell cluster and/or a cell group or a segmented individual cell.

Preferred embodiments of the present invention are defined in the dependent claims.

### BRIEF DESCRIPTION OF THE DRAWINGS

In the following, preferred embodiments of the present invention are explained in more detail with respect to the accompanying drawings, in which:

FIG. 1 shows the steps of the method for the segmentation of a detected cell group according to the inventive method;

FIG. 2A to 2E show the determination of adjacent cell nuclei in a cell group according to a preferred embodiment;

FIG. 3A to 3C show the localization of contractions according to a preferred embodiment of the present invention with respect to two adjacent cell nuclei;

FIG. 4A to 4E show the separation of a common cell plasma according to a preferred embodiment;

FIG. 5A to 5B show the determination of an area of the common cell plasma in which an overlapping of the cell plasmas is expected according to a preferred embodiment;

FIG. 6A to 6E show the completion of a separated cell according to a preferred embodiment; and

FIG. 7 shows another preferred embodiment of the inventive method which includes a detection of cell groups from a sample image.

### DESCRIPTION OF THE PREFERRED EMBODIMENTS

With respect to the following description, it is to be understood that, in the individual figures, elements that are similar or function in the same way are provided with the same reference numbers.

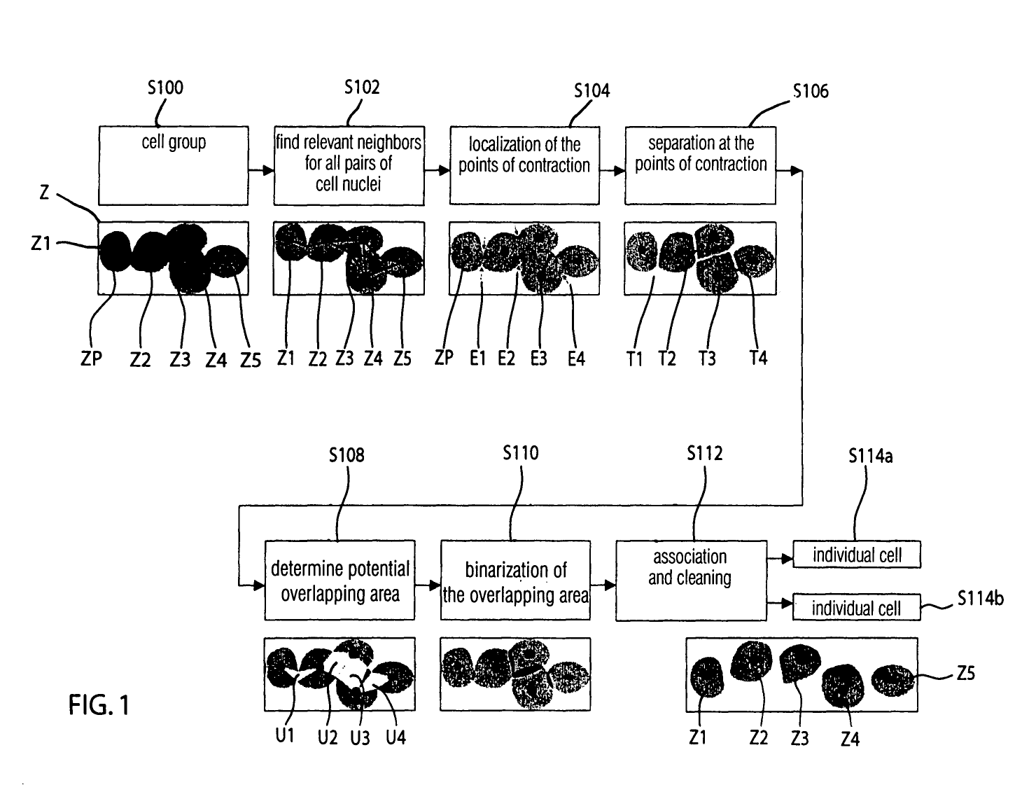

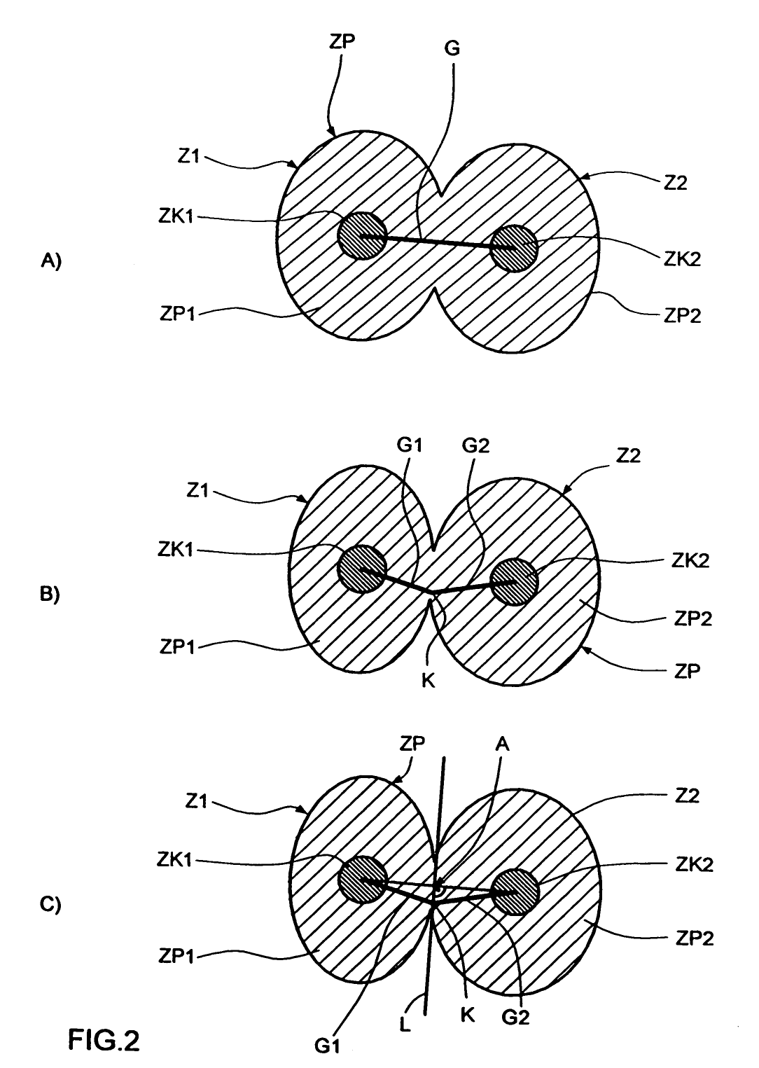

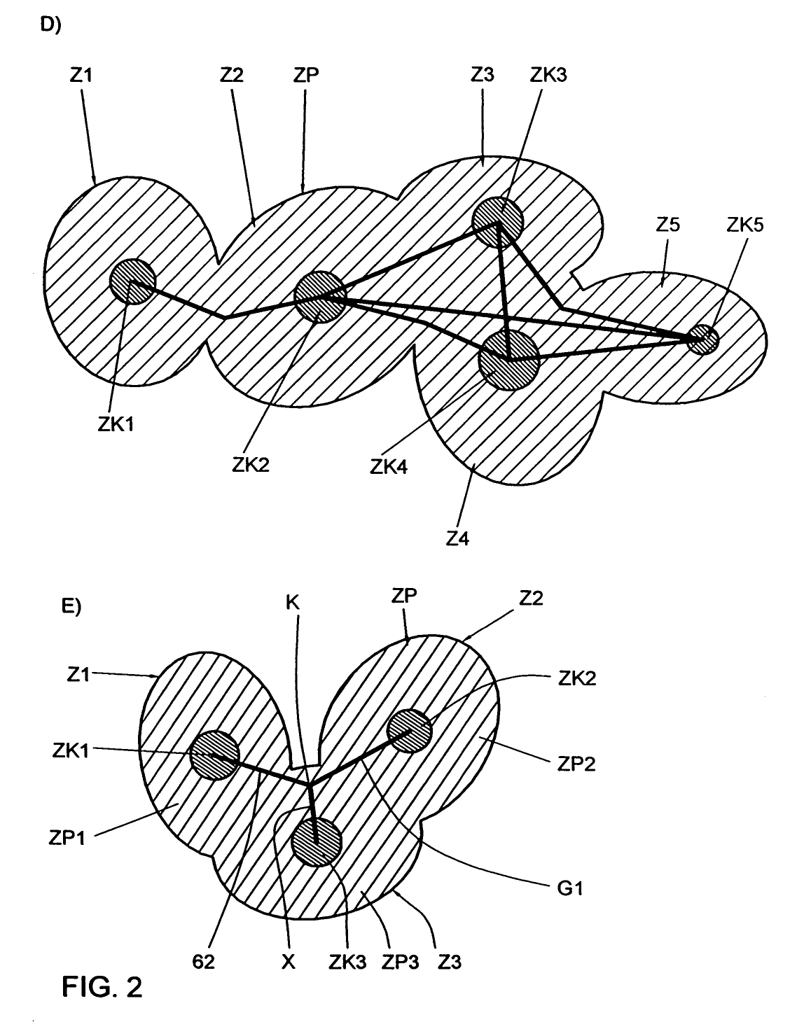

With respect to FIG. 1, a first preferred embodiment of the inventive method is explained in more detail in the following. FIG. 1 shows a block diagram in which, based on an individual cell group detected in a sample image, a segmentation of the cell group into individual cells is performed. Preferred implementations of the individual steps described with respect to the block diagram in FIG. 1 will be explained in more detail with respect to FIGS. 2 to 6 in the following. In the underlying image in FIG. 1, the detectable cell nuclei and cell plasmas have already been determined, based on an original sample image, so that the areas of the cell nuclei and the cell plasmas are given, preferably as binary masks. The detection of the cell nuclei and cell plasmas contained in the original sample image for detecting a cell cluster in the sample image will be described in more detail later on.

FIG. 1 shows the separation of a cell group, a so-called cell cluster, i.e. of cell plasmas with more than one cell nucleus, i.e. mutually overlapping cells, into its components of individual cells. In FIG. 1, on the one hand the individual steps according to the preferred inventive method are explained, wherein each of the individual steps is associated with a diagrammatic representation of the cell cluster after the corresponding step has been performed.

In FIG. 1, the method starts with step S100, in which a cell group Z with a plurality of cells Z1 to Z5 is provided. Each of the cells Z1 to Z5 includes a cell nucleus and a cell plasma. The cell group provided in step S100 is a graphic reproduction of the cell group which is generated from a digitalized picture of a cytological sample to be examined, as will be explained in more detail in the following. The inventive method is based on the image information contained in the picture of the cell group. A modification of the actual cytological sample and/or the prepared cytological specimen is not performed.

After the cell group has been provided in step S100, the method proceeds to step S102, in which relevant neighbors, i.e. relevant adjacent cell nuclei, are detected for all pairs of cell nuclei. An embodiment for the selection or detection of relevant adjacent cells will be described in more detail in the following. In the general embodiment shown in FIG. 1, assume that the cells Z1 and Z2 meet the required criteria for adjacency, i.e. the cell Z2 is adjacent to the cell Z1 to be separated. Also, the cells Z1 and Z3 are adjacent to the cell Z2 to be separated. The cells Z2 and Z4 are adjacent to the cell Z3 to be separated. The cells Z3 and Z5 are adjacent to the cell Z4 to be separated. The cell Z4 is adjacent to the cell Z5 to be separated.

After the adjacent cell nuclei have been determined in step S102, the method proceeds to step S104, in which so-called contraction points are localized. Contraction points or contractions are portions of the common cell plasma formed by all cell plasmas ZP of all cells Z1 to Z5, i.e. portions of the common cell plasma, in which extension of the same compared to the usual extension is low or even minimal. The localization of the contraction points according to step S104 is performed based on an evaluation of the common plasma located between a cell to be separated and a cell adjacent to the cell to be separated. In the embodiment shown in FIG. 1, four contractions E1 to E4 result. The contraction E1 is located between the cell Z1 and the cell Z2 and was determined based on an examination of these two cells. The remaining contractions were also determined by an examination of the adjacent cells.

After the contraction points and/or contractions in the common plasma ZP have been determined, the method proceeds to step S106, in which a separation of the common cell plasma is performed based on the contraction points localized in step S104. As can be seen from FIG. 1, the common plasma was separated at the positions T1 to T4, so that the cells Z1 to Z5 in the picture are now separated from each other.

Although the now individual cells Z1 to Z5 are now separated, they do not correspond to the cells contained at the corresponding positions in the original cytological specimen, because there was an overlapping of the cell plasmas, which has not been taken account of by the simple separation in step S106. For this reason, in step S108, the inventive method determines an area for adjacent cells, in which an overlapping of the cell plasmas of the cells is expected. According to a preferred embodiment, for determining this area, a quadrilateral is subtended which extends from a cell nucleus to a first contraction point, thence to the second cell nucleus, thence to the second contraction point and back to the first cell nucleus. In this area, overlapping cell plasma is expected. In FIG. 1, the overlapping areas defined by the quadrilateral just described are designated U1 to U4.

In step S110, a binarization of the overlapping areas U1 to U5 is performed to associate the pixels of each overlapping area with one or both involved cells by means of a classification step.

In step S112, the cells Z1 to Z5 shown in step S106 are expanded by the overlapping areas associated with the respective cells, and are thus completed to individual cells corresponding to the cells contained in the original cytological sample. Alternatively, cleaning may then be performed in step S112. The thus obtained individual cells are moved a little apart in the picture to separate them clearly from each other.

With respect to the preferred embodiment, it is to be noted that the present invention is, of course, not limited thereto. The present invention allows the separation of a cell cluster or a cell group including two overlapping individual cells in general. Here, too, a determination is made for each cell nucleus which relevant adjacent cells are in the proximity (step S102). Next, the contraction points between the two adjacent cell nuclei are detected (S104), which serve as markers of the cells at which the overlapping ends. The cells are then first separated between the contraction points, and, subsequently, the overlapping area of the cells, subtended by the quadrilateral between the contraction points and the two cells, is determined. The pixels of the overlapping area are associated with one or both cells by means of a classification step. Subsequently, an optional cleaning step is performed, as also shown in FIG. 1.

In the following, preferred embodiments for the implementation of the steps S102 to S112 described in detail with respect to FIG. 1 will be described with respect to FIGS. 2 to 6.

With respect to FIG. 2, the determination of adjacent cell nuclei in a cell group according to a preferred embodiment will be explained in more detail in the following. Examining a cell group that is to be separated into individual cells, the first question arising in the context of separating a cell nucleus is which other cell nuclei—and thus connected cell plasmas—are actually located “in the proximity” so that they have to be considered in a separation of the common plasma. The phrase “in the proximity” represents a simplification. The decision for each of the cell nuclei in a cell group whether it has to be considered or not plays an important role with respect to whether the separated cell plasma area is correct.

According to a preferred embodiment, the question whether an adjacent cell nucleus has to be considered in separating an examined cell nucleus is answered based on a distance existing between the two cell nuclei and whether the two cell nuclei are located in a common cell plasma.

As an example, look at the cells Z1 and Z2 shown in FIG. 2A, each of which have a cell nucleus ZK1 and ZK2. The cells further each have a cell plasma ZP1 and ZP2 which overlap each other, thereby forming a common cell plasma ZP. The allowable distance between the two cell nuclei ZK1 and ZK2 ranges between a maximum distance and a minimum distance. The maximum distance and the minimum distance are determined empirically. In a preferred embodiment, in which the information regarding the individual cells Z1 and Z2 is in binary images, there are so-called binary masks for the individual cell nuclei and, equally, there is a binary mask for the common cell plasma ZP. According to a preferred embodiment, the Euclidean distance between the gravity center of the binary mask of the cell nucleus ZK1 to be separated and the gravity center of the binary mask of the remaining cell nuclei, here of the cell nucleus ZK2, is examined, wherein the distance should be less than 300 pixels. In the embodiment, also the minimum distance is determined which is checked by subtracting from the distance of the gravity centers the average distance of all boundary points from the gravity center of both binary masks. According to the preferred embodiment, the resulting value may not be smaller than 30 pixels. The pixel values just described, as well as the pixel values to be mentioned in the following, apply to images with a resolution of 1000×700 pixels. Depending on the camera, the objective, etc., images with other resolutions may be generated, to which then other pixel values apply.

If another cell nucleus ZK2 meeting the requirements with respect to the distance is adjacent to the cell nucleus ZK1 to be separated, then, in addition, it is necessary to ensure that these two cell nuclei belong to the common cell plasma ZP. In order to determine this, a straight line G, that has to be completely within the common cell plasma of the cell cluster, is drawn between the cell nuclei ZK1 and ZK2 and/or between the gravity centers of the associated binary masks, as shown in FIG. 2A.

If this is not the case, the cell nucleus is not yet discarded, but first a so-called “indirect connection” is checked for. Checking for the indirect connection is described with respect to FIGS. 2B and 2C. Explained in general terms, an “indirect connection” is a connection line formed by two straight lines G1 and G2, wherein the straight line G1 extends from the cell nucleus ZK1 to a common point, the break point K, and wherein the second straight line G2 extends from the second cell nucleus ZK2 to the common break point K, as shown in FIG. 2B. Geometrically, this connection may be described as follows. Between the cell nuclei ZK1 and ZK2 and/or the gravity centers of the associated binary masks, there is a number of points each having the same distance to the cell nuclei. These points are located on a straight line L (see FIG. 2C) perpendicular to the straight line G which connects the cell nuclei ZK1 and ZK2, wherein the straight line L is located on center between the two cell nuclei. All points on the straight line L are now examined individually. For each of the examined points, a straight line G1 starting from the cell nucleus ZK1, and a straight line G2 starting from the cell nucleus ZK2 are formed to the examined point. The subsequent step checks whether both straight lines G1 and G2 are within the common cell plasma ZP. If this is the case, an indirect connection could be established between the gravity centers via these two straight lines G1 and G2.

If the examination of all points reveals that several of these indirect connections exist, the connection selected is the one which has the largest angle between the two straight lines G1 and G2, and/or which is as close as possible to the straight line G representing the closest connection between the cell nucleus, and/or the connection for which both straight lines G1 and G2 are as short as possible. The three conditions stated above are equivalent. The point chosen in the end is the break point K already mentioned with respect to FIG. 2B.

According to a preferred embodiment, what is further provided is that the break point K is only a predetermined distance away from the point A of the normal, in which the straight line L perpendicularly intersects the straight line G. In a preferred embodiment, this maximum distance should be about 50 pixels.

If both the distance condition and the connection condition of two cell nuclei are met, the two cell nuclei are considered to be adjacent to each other. If both or one of the conditions are not met, the cell nucleus ZK1 originally to be separated is not examined any further and is discarded.

Although an example has been described with respect to FIGS. 2A to 2C, the present invention is, of course, not limited thereto, particularly not to the use of two cell nuclei. In FIG. 2D, five cell nuclei ZK1 to ZK5 are shown with their corresponding direct and indirect connections found in the manner described above. The exemplary picture shown in FIG. 2D is an expanded representation of the cell group associated with step S102 in FIG. 1.

In a situation in which there are more than two cell nuclei and in which there is an indirect connection for two adjacent cell nuclei, the break point K described above exists. Now the distances to other cell nuclei are examined for this break point K and a determination is made whether one of these distances is smaller than the distance of the break point to the cell nucleus to be separated, as shown in FIG. 2E. After the determination of the indirect connection between the cell nucleus ZK1 and the cell nucleus ZK2, what was determined here was that the distance X of the break point K to the cell nucleus ZK3 is smaller than the distance of the break point K to the cell nucleus ZK1. The cell nucleus ZK2 originally used for the indirect examination is therefore discarded for the further separation of the cell nucleus ZK1, and the cell nucleus ZK3 takes its place.

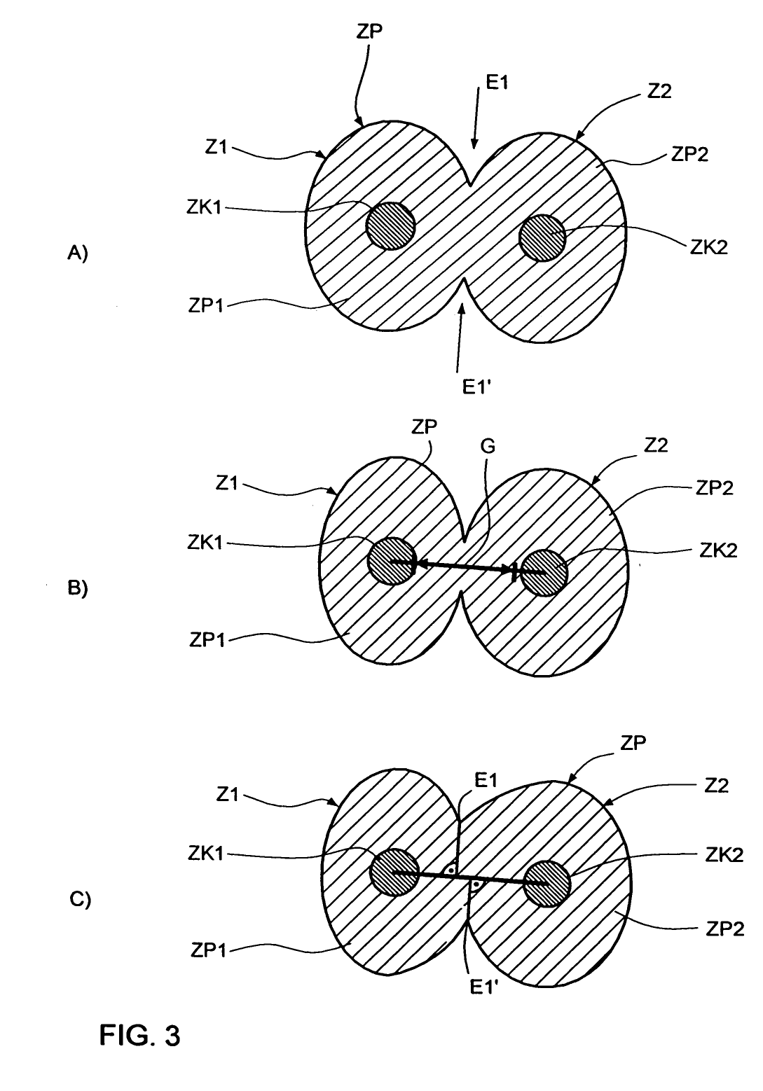

In the following, a preferred embodiment for the localization of contraction points with respect to two adjacent cell nuclei is explained in more detail with respect to FIGS. 3A to 3C. After performing the steps described in more detail with respect to FIG. 2, there are now a list of cell nuclei for the cell nucleus ZK1 to be separated, that are to be considered in separating. In the present embodiment, this list only contains the adjacent cell nucleus ZK2. If it turns out that the list is empty, i.e. there are no cell nuclei adjacent to the examined cell nucleus ZK1 to be separated, this cell nucleus, and above all the associated cell plasma, cannot be separated. This cell nucleus is therefore discarded. This may occur when the current cell nucleus ZK1 is located on the cell plasma of a cell cluster with several cell nuclei, while none of these cell nuclei meets the conditions described above regarding distance and connection. In addition to the cell nuclei entered in the list, the position of the break point K which has been found for the shortest indirect connection between the cell nucleus contained in the list and the cell nucleus to be separated is also given there. If this connection is a direct connection, then the break point is the center point between the examined cell nuclei, in the embodiment as described above between the cell nucleus ZK1 to be separated and the adjacent cell nucleus ZK2.

Based on the cell nucleus to be separated and the adjacent cell nucleus, as well as based on the break point K, now there is a search for contraction points E1, E1′ (see FIG. 3A). According to a preferred embodiment, the contraction points are the most narrow place of the cell plasma ZP connecting the two cell nuclei ZK1 and ZK2, as illustrated in FIG. 3A by the arrows shown there.

In order to find the contraction points, a straight line is drawn through the break point K running parallel to the straight line G between the two cell nuclei. If there is a direct connection between the cell nuclei, then it is the straight line G.

Subsequently, all boundary points of the plasma belonging to the cell group, the common cell plasma ZP, are examined.

For each boundary point, a perpendicular is dropped on the straight line drawn through the break point, and subsequently there is a search for the two boundary points that meet the following conditions.

The first condition is that the points must be “between” the cell nuclei, i.e. the intersection point of the normal must be on the line segment between the cell nuclei. According to another embodiment, this area is further limited by declaring a part of the length of the respective average distance of the boundary points Rn to the gravity center S of the cell nucleus from both ends of the line segment between the cell nuclei as “invalid”, as illustrated in FIG. 3B by the arrow associated with the straight line G.

The second conditions is that one of the sought-for points must be “left” and one of the sought-for points must be “right” of the selected straight line G, as illustrated in FIG. 3C, in which the first point E1 is located above the straight line G and the second point E1′ is located below the straight line. When determining the perpendicular, this can be seen from positive or negative signs, respectively.

The last condition is that, on both sides of the straight line G, the point with the shortest perpendicular is chosen.

If all these conditions are met, the contraction points E1 and E1′ between the cell nucleus ZK1 to be separated and the adjacent cell nucleus ZK2 are determined. If no boundary points are found which satisfy the conditions stated above, the method for the examined cell nucleus ZK1 is stopped, because no appropriate position for a separation has been found.

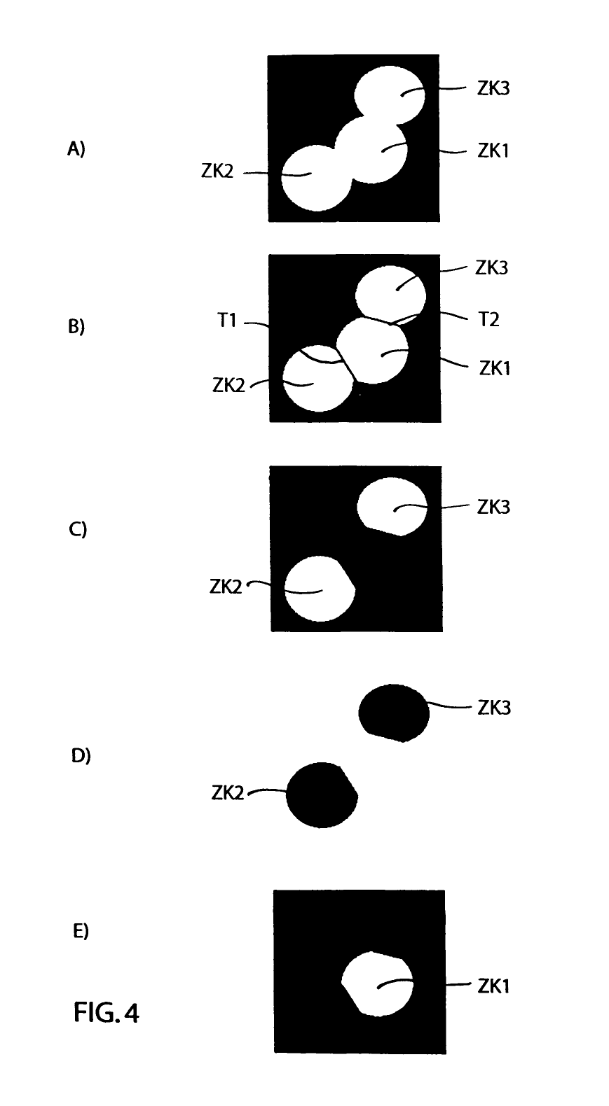

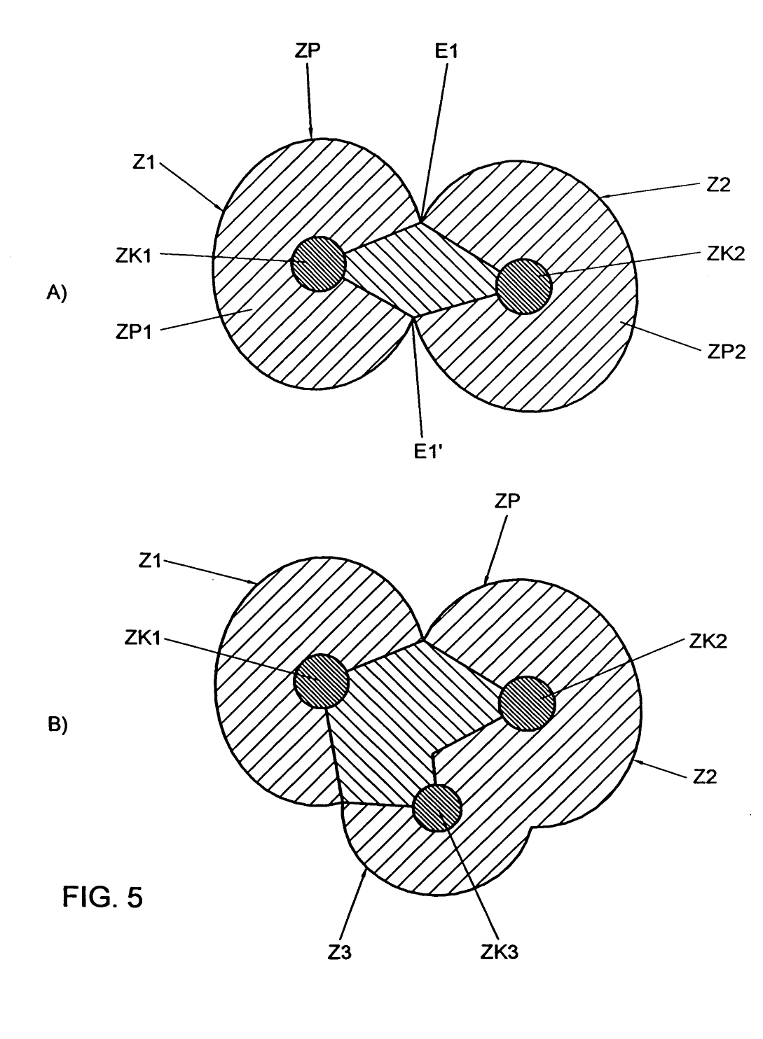

After the contraction points have been found, now a preferred embodiment for the separation of the common cell plasma is explained in more detail with respect to FIG. 4.

According to a preferred embodiment, the detected contraction points are added to the existing list of relevant cell nuclei. Based on the contraction points, a straight line is drawn between the same between each pair consisting of the cell nucleus to be separated and a cell nucleus which is filed in the list and which is relevant because it is adjacent, and the common cell plasma of the cell group is “cut off” at this straight line. This cut is performed to obtain a rough basis for the area of the common cell plasma to be separated.

According to a preferred embodiment containing the information regarding the common cell plasma and the cell nuclei in a binary mask containing “white” and “black” pixels, the cutting-off is performed by drawing a “black” line between the contraction points of a pair, which is performed for all contraction points.

In FIG. 4A, a binary mask of a cell group is shown, wherein three cell plasmas that cannot be detected in the binary mask are to be separated from each other. In FIG. 4A, only the contours of the common cell plasma ZP can be detected. For convenience, the individual portions of the binary mask are designated ZK1, ZK2, ZK3 in FIG. 4A. Only the separating of the portion ZK1 is looked at. The algorithm works such that straight lines T1, T2 are drawn at the contraction positions to separate the individual portions from each other. Subsequently, the area to be cut out is filled so that the binary mask shown in FIG. 4C is established which is subsequently inverted, as shown in Fig. C. A Boolean intersection operation of the binary mask shown in FIG. 4D with the original binary mask of FIG. 4A leads to the binary mask in FIG. 4E which only contains the portion of the common cell plasma to be separated from the cell group.

Before a potential overlapping area is subsequently determined, the distance between the two contraction points is examined according to a preferred embodiment. If this distance is below a predetermined, empirically determined threshold value, for example 40 pixels, it is to be assumed that the cell plasmas of the adjacent cell nuclei only touched at this section, but did not really overlap. If such a situation is detected, no further processing is required, but the separated portion actually shows the cell that was in the original sample.

Further there is an examination whether the distance of the contraction points does not exceed a maximum value, such as 350 pixels. If exceeding of the maximum value is detected, it is to be assumed that the determination of the contraction points was not done correctly, because an overlapping of this length is unlikely or the cell cluster must be considered indivisible, at least at this place. In this situation, the separation of the cell nucleus of interest with cell plasma is then stopped.

After the rough basis of the cell plasma has been separated from the cell group, it is principally assumed that, at each of the sections, an overlapping of cell plasmas of various cells had occurred. Therefore, in a subsequent step, an area has to be determined in which this overlapping of the plasmas is to be looked for.

With respect to FIGS. 5A and 5B, a preferred embodiment is described by way of which an area of interest is determined in which overlapping of the cell plasmas of the cells contained in the original sample is expected.

As shown with respect to FIG. 5A, a quadrilateral is formed with the two contraction points E1, E1′ and the two cell nuclei ZK1 and ZK2, which generally has the form of a rhombus. Within this quadrilateral, the overlapping of the cell plasmas in the original sample is expected. The inner area of the quadrilateral is again represented as a binary mask according to a preferred embodiment, and it is intersected with the binary mask of the common cell plasma of the cell group in a Boolean fashion, because the overlapping can, of course, only occur within the plasma, and therefore no pixel outside the plasma is to be examined. This is necessary because, of course, parts of the quadrilateral may be located outside the plasma. Subsequently, the binary mask of the involved cell nuclei is subtracted from the resulting binary mask, because also the areas of the cell nuclei are not used for the detection of the overlapping areas of the cell plasmas.

It may happen in a cell group that several adjacent cell nuclei exist for the cell nucleus to be cut out. For each one of them, the contraction points are determined and potential overlapping areas are formed. If it happens that two or more of these potential overlapping areas intersect, they are combined to a single binary mask and treated together. With respect to FIG. 5B, such a situation is shown, wherein the overlapping area is represented in a hatched manner.

As was explained above, the overlapping of the cell plasmas of the individual cells contained in the original sample is expected within the overlapping area. This can generally be seen, for example, by a darker chrominance in a transmitted light image of the sample, because two overlapping plasmas appear darker than one plasma. The easiest way to solve this distinction is with a histogram and an appropriate threshold value determination.

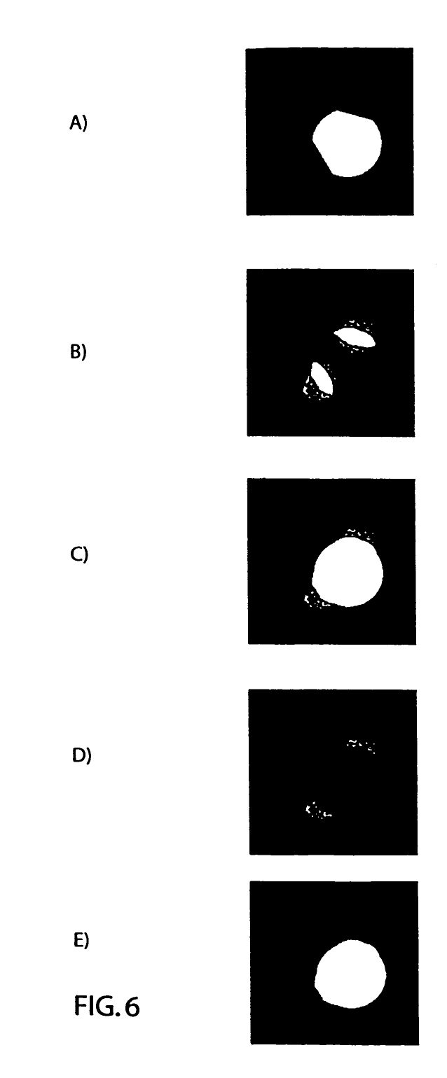

According to a preferred embodiment, now a local histogram of the generated image, such as the transmitted light image, is established with respect to the determined area of overlapping. Subsequently, the histogram is examined in order to determine a threshold value and, with this value, binarize the generated image within the bit mask. This examination may, for example, be performed using the method of Otsu which is described in more detail by T. Lehmann, W. Oberschelp, E. Pelikan, and R. Repges in “Bildverarbeitung für die Medizin”, Springer, Berlin 1997. In this way, the darker pixels in the overlapping are represented white and the brighter pixels in the overlapping are represented black in the binary mask. This is illustrated in FIGS. 6A and 6B, wherein FIG. 6A shows the rough mask for the cell plasma previously described. FIG. 6B shows the overlapping binary mask resulting due to the steps described above.

This overlapping binary mask is combined with the binary mask of FIG. 6A, resulting in the binary mask shown in FIG. 6C. The artifacts still present at the boundary are cleaned so that the final form results as shown in FIG. 6E.

In the manner described above, cell groups in a specimen may thus be split up into individual cells by means of the inventive method so that, by the automatization at this point, an overall automatization of the classification method for cytological specimens is achieved.

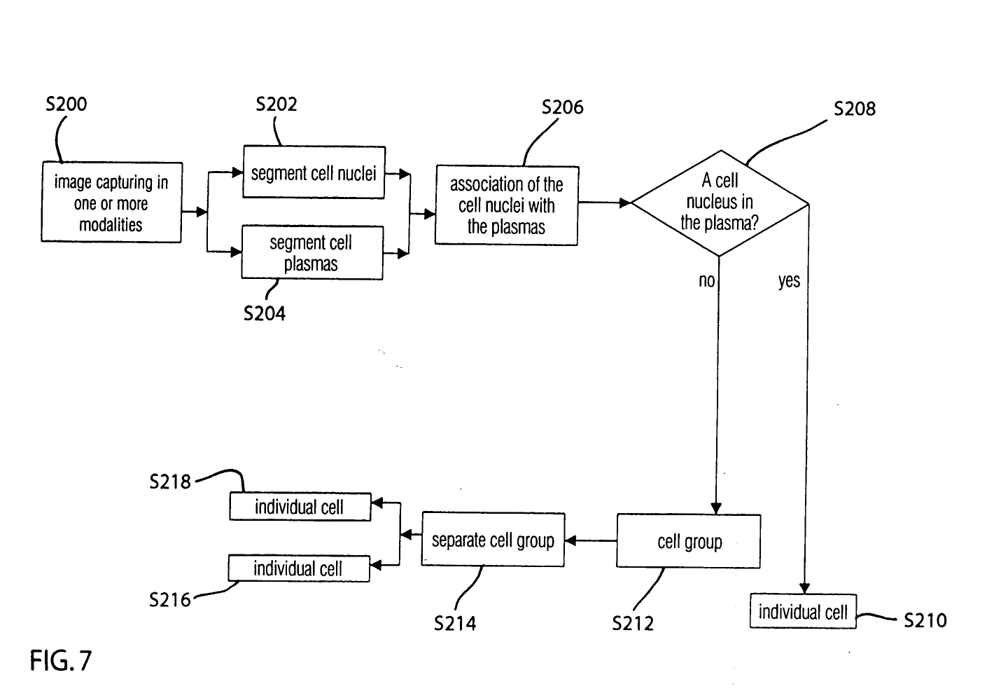

As has been mentioned above, the inventive method starts with a cell cluster and/or a cell group detected from a picture of a cytological sample. In the following, a block diagram of another preferred embodiment of the present invention is described with respect to FIG. 7, according to which the method includes the necessary steps for the preparation of a cell group.

In this embodiment, the method starts with step S200, in which capturing an image of the cytological sample is performed in one or more modalities. As has already been mentioned above, capturing an image is either performed with a capturing modality, such as transmitted light or fluorescence. Alternatively, several multi-modal images registered with each other may be generated, for example by generating images of a sample in a first capturing modality and a second capturing modality. The first capturing modality may, for example, be a transmitted light capturing modality, and the second capturing modality may be a fluorescence capturing modality. Alternatively, fluorescence capturing modalities with different parameters may also be employed.

In the subsequent step S202, the cell nuclei in the picture are detected and segmented to generate a list of the cell nuclei contained in the image and/or the picture. In parallel, the detection of the cell plasmas contained in the picture and their segmentation are performed in step S204 to generate, in turn, a list containing the cell plasmas in the picture. It is to be noted that, when using several images, the segmentation of cell nuclei and the segmentation of cell plasmas does not have to be performed in the same images. Preferably, the segmentation of cell plasmas will be performed on the basis of transmitted light images, whereas the segmentation of cell nuclei may be performed on the basis of fluorescence images. After the cell nuclei and cell plasmas in the sample have been detected, the cell nuclei are associated with the plasmas in step S206, via the generated lists. Subsequently, there is an examination in step S208 whether a plasma is associated with only one single cell nucleus. If this is the case, then this is an individual cell that does not require further segmentation, and the method ends with step S210. If a plasma is detected to be associated with more than one cell nucleus, the method proceeds to step S212 in which the presence of a cell group is detected. This cell group is subsequently separated in step S214 so that, finally, there are the individual cells in the steps S216 and S218 for further processing. With respect to the steps performed in step S214, see the above description of the preferred embodiment for cell group separation.

In the following, a preferred embodiment for detection and segmentation of the cell plasmas and cell nuclei in the picture of a cytological sample will be described.

The cell plasma segmentation is optionally performed in a transmitted light image or in a fluorescence image of the sample. The cell plasma segmentation is performed using histograms. Here, a predetermined threshold value is calculated (e.g. by the method of Otsu mentioned above), with which the transmitted light image is binarized to thus separate cell plasmas from the brighter background. For forming the histograms and the threshold values, various methods well known in the art are implementable.

The binary image of the picture of the cell generated by the histogram-based approach is now examined to determine regions in the binary image which reproduce the plasma, including nucleus, of a cell or which reproduce a cell cluster of overlapping cells. Each independent area in the binary image represents a region of its own, and a sub-image, e.g. in the form of a binary mask, is associated with each individual region, i.e. with each plasma of the cell and/or each area of a cell cluster of overlapping cells.

The cell nucleus segmentation is performed in a similar manner to the segmentation of the cell plasmas, optionally in the transmitted light image or in the fluorescence image. Here, the known histogram-based approach for the detection of cell nuclei in the picture of the cytological sample is also used, so that sub-images, e.g. in the form of binary masks, result for individual cell nuclei.

The list of sub-images (binary masks) resulting from the segmentation of the cell plasmas, wherein each sub-image corresponds to the plasma of a cell and/or the area of a cell cluster of overlapping cells, and the sub-images of involved cell nuclei resulting from the segmentation of the cell nuclei are combined by means of a simple Boolean operation. If the intersection of the binary masks of the cell nucleus and the binary mask of a cell plasma is not empty, then the cell nucleus is associated with the cell plasma. If a cell plasma is detected to be associated with only one cell nucleus, then these are already completely segmented cells with a plasma and a cell nucleus. If a plasma is associated with two or more cell nuclei, then there is a cell cluster or a cell group that is to be separated according to the invention.

In an embodiment of the present invention, a classification of the cell nuclei is performed based on the sub-images associated with the detected cell nuclei, which includes a comparison of selected parameters of the cell nucleus with predetermined parameters in order to determine whether a detected cell nucleus is suitable for further processing.

Based on the picture of the cytological sample thus prepared and processed, the inventive method performs the division of the segmented cell clusters into individual cells.

The above description of the preferred embodiment has set forth that the overlapping area is formed by a quadrilateral. The present invention is not limited to this implementation, the overlapping area may rather be subtended by an area of any form between the contraction points E1, E1′ and the cell nuclei ZK1 and ZK2.

While this invention has been described in terms of several preferred embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations, and equivalents as fall within the true spirit and scope of the present invention.

1. A method for separating a cell group contained in an image of a sample into individual cells for a subsequent classification of the sample, wherein the cell group comprises a plurality of mutually overlapping cells, the method comprising:
(a) selecting a cell nucleus of a first cell which is to be separated from the cell group, wherein the cell nucleus of the first cell is located adjacent to a cell nucleus of a second cell, wherein the cell plasma of the first cell and the cell plasma of the second cell overlap each other such that a common cell plasma is formed;
(b) determining a contraction of the common cell plasma between the cell nucleus of the first cell and the cell nucleus of the second cell;
(c) separating the common cell plasma at the contraction;
(d) determining an area of the common cell plasma in which the overlapping of the cell plasmas of the first cell and the second cell is expected;
(e) classifying the determined area to associate individual portions of the same with the cell plasma of the first cell and/or the cell plasma of the second cell; and
(f) completing the cell plasma of the first cell obtained in step (c) based on the classified portions.

2. The method of claim 1, wherein step (a) comprises the following steps:
(a.1.) determining a distance between the cell nucleus of the first cell and the cell nucleus of the second cell;
(a.2) determining whether the cell nucleus of the first cell and the cell nucleus of the second cell are located within the common cell plasma;
(a.3.) if, in step (a.1.), the distance is detected to be outside a predetermined area, and/or if, in step (a.2.), the cell nuclei are detected not to be located within the common cell plasma, classifying the cell nuclei as not adjacent; and
(a.4.) if, in step (a.1.), the distance is detected to be within the predetermined area, and if, in step (a.2), the cell nuclei are detected to be located within the common cell plasma, classifying the cell nuclei as adjacent.

3. The method of claim 2, wherein, in step (a.2), it is detected whether the cell nucleus of the first cell and the cell nucleus of the second cell are connected by a straight line running completely within the common cell plasma, or whether, between the cell nucleus of the first cell and the cell nucleus of the second cell, there is a common point to which the cell nuclei have the same distance and which is located in the common cell plasma.

4. The method of claim 3, wherein the cell nucleus of the first cell and the cell nucleus of the second cell are classified as not adjacent, if another cell with a cell nucleus exists whose distance to the common point is smaller than the distance of the cell nucleus of the second cell to the common point.

5. The method of claim 1, wherein step (c) includes the following step in order to determine two contraction points of the common cell plasma:
for all boundary points of the common cell plasma located between the cell nuclei, determining the distance of each boundary point to a determined straight line and selecting the boundary points as contraction points which have a predetermined distance to the straight line.

6. The method of claim 5, wherein the predetermined distance is a minimum distance of all examined boundary points.

7. The method of claim 1, wherein, in step (c), the first cell is separated from the cell group along a separating line determined by the contraction.

8. The method of claim 1, wherein step (d) includes the following step:
determining the area located between the cell nucleus and the contraction points.

9. The method of claim 1, wherein, in step (e), the determined area is classified based on the transparency of the common cell plasma in the individual portions.

10. The method of claim 1, wherein at least one further cell in the cell group is located adjacent to the first cell, and wherein the steps (b) to (e) are also performed for the first cell and the further cell.

11. The method of claim 1, wherein the image of the sample is generated by a picture of the sample in a transmitted light capturing modality, or wherein the image includes a plurality of sub-images which are registered with each other and which were generated in the same or in different capturing modalities.

12. The method of claim 1, wherein the method includes the following steps prior to step (a):
detecting and segmenting cell nuclei in an image of the sample to generate a list of cell nuclei;
detecting and segmenting cell plasmas in the image to generate a list of cell plasmas;
associating the cell nuclei with the pertinent cell plasmas; and
detecting cell plasmas associated with more than one cell nucleus to determine a cell group for a subsequent separation.

13. The method of claim 12, wherein the step of detecting and segmenting cell nuclei is based on an image of the sample generated in a transmitted light capturing modality or a fluorescence capturing modality, wherein cell nuclei in the image are detected based on a histogram and a predetermined threshold value, and wherein a sub-image is associated with each detected cell nucleus.

14. The method of claim 13, wherein, based on the sub-images associated with the detected cell nuclei, a classification of the cell nuclei is performed which includes a comparison of selected parameters of the cell nucleus with predetermined parameters in order to determine whether a detected cell nucleus is suitable for further processing.

15. The method of claim 13, wherein the step of searching for and segmenting cell plasmas is based on an image of the sample generated in a transmitted light capturing modality or in a fluorescence capturing modality, wherein cell plasmas in the image are detected based on an edge detection or based on a histogram and a predetermined threshold value, wherein a sub-image is associated with each detected cell plasma.

16. The method of claim 12, wherein the image of the sample is binarized in the search for and segmentation of cell nuclei and cell plasmas, and wherein the sub-images are formed by binary masks.

17. The method of claim 13, wherein the step of associating cell nuclei with pertinent cell plasmas and detecting cell groups includes the comparison of the sub-images generated for the cell nuclei and the cell plasmas, wherein, depending on the comparison, one or more cell nuclei are associated with a cell plasma, wherein the sub-images associated with each other are associated with a first group containing sub-images of a cell plasma and one cell nucleus and a second group containing sub-images of a cell plasma and a plurality of cell nuclei, wherein the sub-images of the second group indicate a cell group on which the further processing is based.
